(19) United States
(12) Patent Application Publication
(10) Pub. No.: US 2023/0040306 A1
(43) Pub. Date: Feb. 9, 2023

(54) SYSTEM FOR SELECTIVE REMOVAL OF A TARGET SUBSTANCE FROM A BIOLOGICAL FLUID

(71) Applicant: Maco Pharma, Mouvaux (FR)

(72) Inventors: Quentin BREBANT, Mouvaux (FR); Timothee HOUSSIN, Mouvaux (FR); Louis PAQUET, Mouvaux (FR); Rosa NICOLAS, Mouvaux (FR)

(21) Appl. No.: 17/758,633

(22) PCT Filed: Jan. 14, 2021
(86) PCT No.: PCT/EP2021/050728

§ 371 Date: Jul. 12, 2022

(30) Foreign Application Priority Data
Jan. 14, 2020 (FR) FR2000316

(51) Int. Cl. A61M 1/02

(52) U.S. Cl. CPC A61M 1/0218; A61M 1/0231; A61M 2202/0439; A61M 2205/7527

(57) ABSTRACT

The invention relates to a system for treating a biological fluid such as blood or a blood component by selective removal of a target substance, comprising a treatment bag (2) provided with at least one inlet orifice (3) and at least one outlet orifice (4), said treatment bag containing a set of particles (5) having an affinity for the target substance, said set comprising small particles having a size of less than 15 μm, said system further comprising a barrier means (6) composed of at least one porous membrane made of a hydrophilic material, the pores of said membrane being calibrated to a size suitable for preventing the passage of said small particles, namely less than 3 μm.

The present invention relates to a system for treating a biological fluid such as blood or a blood component by selective removal of a target substance, as well as to a method for treating a biological fluid using such a system.

The invention is typically applicable to the treatment of biological fluids and in particular blood, blood components or blood derivatives intended to be transfused into a patient and where the target substance includes viruses, proteins such as prion proteins, bacteria, parasites, cells such as leukocytes, tumour or cancer cells, toxins, surface or circulating antigens, antibodies, or endogenous or exogenous substances such as exogenous substances used in a pathogen inactivation process. It is particularly applicable to the selective removal of anti-A and/or anti-B antibodies from blood or plasma.

A filtration unit for the selective removal of a target substance present in the blood comprising particles having an affinity for the target substance is known from WO2007/042644. These spherical particles have in particular an average diameter of between 20 and 150 μm and are held in the filter unit between two layers of non-woven fabric having an average pore diameter of less than 8 μm.

In document U.S. Pat. No. 7,700,746, a filtration column used to reduce the amount of anti-A and anti-B antibodies in whole blood or blood plasma is described. In particular, the column comprises particles bound via a spacer to a saccharide such as a blood group A or B determinant. For blood filtration, the particles have a size of between 100 and 250 μm and are held in the column with the aid of a membrane having a porosity of between 30 and 100 μm.

In this type of particle device, it is important to ensure that the particles do not contaminate the filtered fluid. The devices described above are suitable for purifying blood using particles with a calibrated size greater than about 20 μm, but are not suitable where the average particle size is less than about 15 μm or where the particle size distribution is such that a significant proportion of the particles, for example 10%, have a size less than 15 μm. The use of a lower porosity material to constrain these small particles in such devices may impede the proper flow of fluid through such devices and/or may alter the quality of the biological fluid by retaining components of interest.

In document US 2012/0219633, a system for sequentially removing immunoglobulins and leukocytes from blood is described. The system comprises a bag containing immunoglobulin-binding particles and a porous, fibrous filter for retaining leukocytes. The particles are retained by a mesh or sieve bag, that is to say a material with large openings, formed by a network of metal or plastic wires, similar to a net. For example, particles are retained by a polyethylene sieve with openings of 35-40 μm. The openings of these sieves are too large to be able to retain leukocytes.

The invention proposes a system for the selective removal of a target substance present in blood or a blood component using particles which may be small in size, in particular less than 15 μm, enabling volumes of blood of up to two litres to be treated in a reasonable time, while maintaining the biological properties of the blood and ensuring that the treated blood is free of particles.

To this end and according to a first aspect, the invention proposes a system for treating a biological fluid such as blood or a blood component by selective removal of a target substance, comprising a treatment bag provided with at least one inlet orifice and at least one outlet orifice, said treatment bag containing a set of particles having an affinity for the target substance, said set comprising small particles having a size of less than 15 μm, the system further comprising a barrier means for isolating said particles from the biological fluid, said barrier means being composed of at least one porous membrane made of a hydrophilic material, the pores of said membrane being calibrated to a size suitable for preventing the passage of said small particles, namely less than 3 μm.

According to a second aspect, the invention relates to a method for treating a biological fluid such as blood or a blood component by selective removal of a target substance using a system according to the first aspect, said system further comprising a transfer bag in fluid communication or intended to be put in fluid communication with the treatment bag via a transfer tubing, said method comprising the steps of:
introducing the biological fluid to be treated into the treatment bag containing the set of particles having an affinity for said target substance,
contacting said biological fluid with said set of particles for a time sufficient to bind the target substance to said particles, so as to obtain a mixture of treated biological fluid from which the target substance has been removed and particles bound or not to the target substance,
transferring said mixture from the treatment bag to the transfer bag via the transfer tubing, the barrier means of said system preventing said set of particles from passing into the transfer bag, so as to obtain the biological fluid thus treated separated from said set of particles, and
collecting the biological fluid thus treated and separated from the set of particles in the transfer bag 7.

The annexed drawings illustrate the invention:

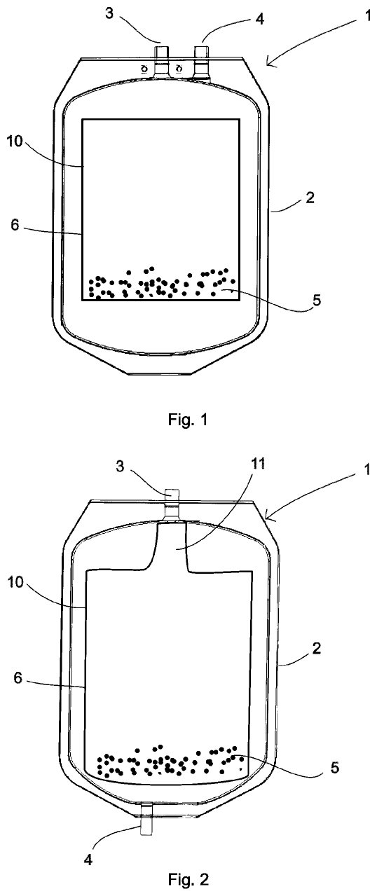

FIG. 1 schematically shows a treatment bag of a system according to an embodiment of the invention in which the barrier means is loosely arranged in said treatment bag.

FIG. 2 schematically shows a treatment bag of a system according to another embodiment of the invention in which the barrier means is fixed in said treatment bag.

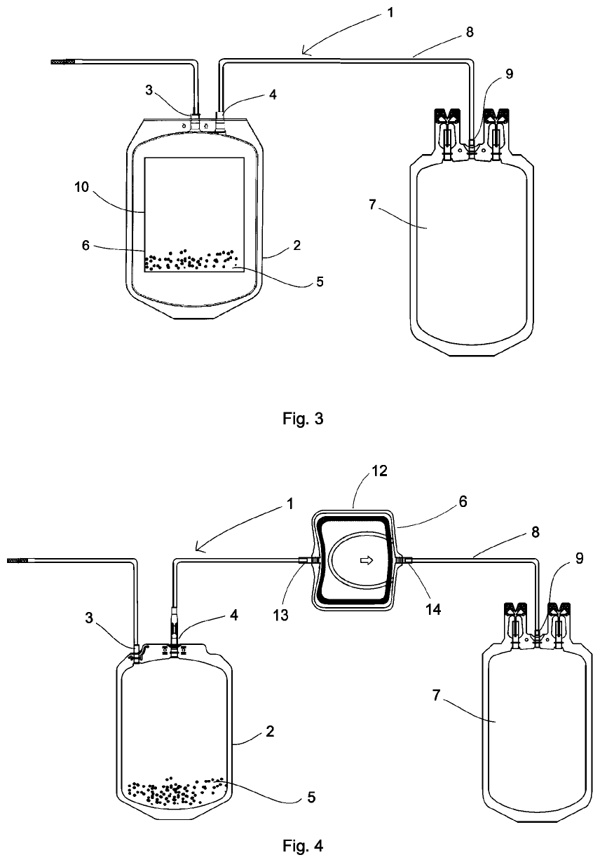

FIG. 3 schematically shows a system according to a particular embodiment comprising the treatment bag from FIG. 1 and a transfer bag.

FIG. 4 schematically shows a system according to a further embodiment of the invention comprising a treatment bag, a transfer bag and a barrier means arranged in a filter unit disposed between said treatment bag and said transfer bag.

According to a first aspect, the invention provides a system for treating a biological fluid by removing a target substance. In particular, the biological fluid is blood or a blood component to be transfused. The blood component is in particular a red blood cell concentrate, a platelet-rich plasma, a low-platelet plasma, or a platelet concentrate.

In some cases, it is necessary to remove a target substance from blood or a blood component prior to transfusion to a patient.

For example, the target substance includes leukocytes, prion proteins, viruses, bacteria, parasites, fungi or other pathogens.

In another example, the target substance is an anti-A antibody and/or an anti-B antibody. By removing anti-A and anti-B antibodies from a donor's plasma, it can be made compatible with patients of any blood type.

In yet another example, the target substance is a substance used to inactivate pathogens in blood or a blood component. Such a substance is for example methylene blue, a psoralen derivative, or riboflavin.

The removal of the target substance is complete or partial, that is to say sufficient to remove or reduce the infectious risk from the infectious agents, and/or remove or reduce the toxicity of the target substances, to a level acceptable for transfusion.

With reference to the drawings in FIGS. 1 to 4, the system 1 comprises a treatment bag 2 provided with at least one inlet orifice 3 and at least one outlet orifice 4. This treatment bag 2 is intended to contain the biological fluid to be treated.

The treatment bag is made from two flexible sheets joined together around their periphery to form an inner volume. The interior volume is sufficient to contain the fluid to be treated. For example, the treatment bag is configured to contain a quantity of biological fluid of between 20 ml and 2000 ml, in particular between 100 ml and 700 ml.

The treatment bag contains a set of particles 5 having affinity for the target substance. Removal of the target substance is thus achieved via the particles that are capable of affinity binding to the target substance. The particles bound to the target substance are then separated from the biological fluid.

For example, the particles have an affinity for one type of target substance. Alternatively, the particles have an affinity for more than one target substance to enable simultaneous removal of more than one target substance from a biological fluid.

In one particular example, the particles comprise a mixture of particles having an affinity for anti-A antibodies and particles having an affinity for anti-B antibodies.

In particular, the particles are adsorbent particles, such as activated carbon particles, aluminium oxide, and silica. Alternatively, the particles are based on polymer such as polystyrene, polycarbonate, cellulose, dextran, polymethacrylate or polyacrylate.

In particular, these particles are physically and/or chemically treated to improve their specificity and/or their affinity for the target substance(s).

More particularly, the particles are grafted with oligosaccharides, such as oligosaccharides capable of binding antibodies such as anti-A antibodies and/or anti-B antibodies. For example, these particles are ultrafine cellulose particles grafted with an antigenic group A or B oligosaccharide, such as those described in document WO2016/177967. In another example, the particles are those described in EP3141558A1 or the IsoClear beads marketed by Prometic Bioseparations.

The set of particles contained in the treatment bag comprises small particles having a size of less than 15 μm.

In a first example, all particles are small particles having an average size of less than 15 μm; more particularly less than 10 μm.

In another example, the particles have an average size greater than 15 μm, but a portion of the particles are small, namely less than 15 μm, in particular less than 10 μm.

The set of particles can be characterised by the particle size distribution. This measurement of the particle size distribution is in particular carried out by means of a laser diffraction granulometer.

In particular, the set of particles comprise 10% of the particles with a size less than 15 μm, in particular less than 10 μm.

The particles are generally substantially spherical and their size corresponds to the diameter of the sphere. In the case where the particles are not spherical, their size corresponds to the equivalent diameter, that is to say the diameter of the sphere of the same volume which would behave identically to the non-spherical particle when retained.

In one particular example, the particles are ellipsoidal in shape with approximate dimensions of 2.2 μm by 8 μm. These particles are considered to be approximately 2 μm in size.

The set of particles 5 is generally left free in the treatment bag. In a variant, in order to avoid their agglomeration, the set of particles is impregnated or integrated into a textile layer such as a non-woven fabric, as envisaged in document WO2007/042644.

According to a particular embodiment, the amount of particles ranges from 1 to 10 mg/ml of biological fluid, in particular from 2 to 5 mg/ml of biological fluid.

According to the invention, the system further comprises a barrier means 6 which blocks the passage of particles but allows the passage of biological fluid.

This barrier means 6 is composed of at least one porous membrane made of a hydrophilic material, the pores of said membrane being calibrated to a size suitable for preventing the passage of said particles, namely less than 3 μm.

This porous membrane is planar and has a non-crosslinked structure, in particular without entanglement of threads, fibres or strands, and no net structure. This porous membrane is for example manufactured by phase inversion.

The pores of the membrane have a size suitable for preventing the passage of said particles, that is to say the pore size of the membrane is substantially smaller than the minimum particle size.

In particular, the porous membrane has a thickness between 90 μm and 150 μm.

In a particular embodiment, to ensure that the set of particles does not include particles having a size smaller than that of the pores of the porous membrane, the set of particles is passed through a sieve having pores with a size corresponding to that of the pores of the porous membrane. The particles that are not passed through the sieve form the set of particles of the system for treating the biological fluid.

In particular, the pores of the membrane are calibrated to a size less than or equal to 3 μm, in particular less than or equal to 1 μm. For example, the pores of the membrane are approximately 0.65 μm.

This membrane with a porosity of approximately 0.65 μm is advantageous in that it also allows the simultaneous retention of cellular constituents such as leukocytes from the blood plasma.

The membrane is made of a hydrophilic material selected from naturally hydrophilic materials or plastics-based materials that have been made hydrophilic. Indeed, the hydrophilic nature of the material is important to ensure good haemocompatibility with blood or blood components.

For example, the hydrophilic material is selected from polymers and/or copolymers based on polypropylene, polyester, polyamide, high- or low-density polyethylene, polyurethane, polyvinylidene fluoride, or cellulose-based products such as cellulose acetate and its derivatives.

These polymeric products are generally not naturally hydrophilic and must be treated by physical or chemical methods, to impart said hydrophilic properties.

These treatments consist, for example, in grafting hydrophilic substituents, for example hydroxyl or carboxylic groups, onto the polymer, according to known methods. Another treatment consists in carrying out a gaseous plasma treatment, in particular oxygen plasma treatment.

In relation to FIGS. 3 and 4, the system further comprises a transfer bag 7 in fluid communication or intended to be put in fluid communication with the treatment bag 2 via a transfer tubing 8. This transfer bag 7 is intended to collect the treated biological fluid, depleted in target substance and free of particles.

The transfer tubing 8 is connected to the outlet orifice 4 of the treatment bag 2 and to an inlet orifice 9 of the transfer bag 7.

The barrier means 6 is disposed inside or outside the treatment bag 2.

In an embodiment shown in FIGS. 1 to 3, the barrier means 6 is disposed inside the treatment bag 2. In this case, the barrier means is intended to retain the particles in the treatment bag 2. During transfer of the treated biological fluid from the treatment bag 2 into the transfer bag 7, the particles 5 are confined within the treatment bag 2. The biological fluid collected in the transfer bag 7 is thus free of particles.

In a first configuration shown in FIG. 1, the barrier means 6 is in the form of a pouch 10 enclosing the particles 5, said pouch being composed of said porous membrane.

The porosity of the porous membrane forming the pouch is such that particles 5 cannot exit the pouch 10 through its walls, but biological fluid can pass through it to contact these particles 5.

The pouch is made, for example, by joining on the sides a porous membrane placed in duplicate or joining on the bottom and sides two membranes placed one above the other. The connection is made in particular by thermal, high-frequency, or ultrasonic welding.

In one variant, the pouch 10 is a double-walled pouch, each of the walls being composed of the porous membrane. In this variant, the double-walled pouch is produced by, for example, arranging a first pouch containing the particles in a second pouch, each of the first and second pouches comprising the porous membrane. Thus, if small particles escape from the first pouch, they are retained by the porous membrane of the second pouch.

In another embodiment, the pouch 10 includes first and second peripheral seals spaced apart from each other. Thus, if one of the peripheral seals is defective, the other peripheral seal seals the pouch 10.

In order to ensure good containment of the particles in the pouch 10, the pouch is a double-walled pouch with a double seal.

In FIG. 1, the pouch 10 is sealed, that is to say the top of the pouch is closed, in particular by welding. The particles are then enclosed within the pouch 10.

Advantageously, the pouch 10 is loose in the treatment bag 2. Alternatively, it is attached by one side to one edge of the treatment bag 2.

In an alternative configuration shown in FIG. 2, the pouch 10 containing the particles 5 is arranged within the treatment bag 2 and has an opening 11, said opening being in fluid communication with the inlet orifice 3 of the treatment bag 2.

In this configuration, the biological fluid to be treated is introduced into the pouch 10 of the treatment bag 2 to be contacted with the particles, and then passes through the porous membrane forming the pouch.

In a further configuration (not shown), the barrier means 6 is in the form of a wall composed of said porous membrane, said wall being arranged within the treatment bag 2 so as to delimit an inlet compartment in fluid communication with the inlet orifice 3 of the treatment bag and an outlet compartment in fluid communication with the outlet orifice 4 of the treatment bag 2. The inlet compartment of the treatment bag 2 encloses the particles 5.

Advantageously, the wall comprising the porous membrane is held in a sealed frame, which facilitates the assembly of the barrier means 6 in the treatment bag 2.

In another embodiment shown in FIG. 4, the barrier means 6 is disposed outside the treatment bag 2, in the flow path defined by the transfer tubing 8.

The barrier means 6, external to the treatment bag 2, is for example in the form of a filter unit 12, said filter unit enclosing a filtration medium composed of at least said porous membrane.

In this embodiment, the system comprises a transfer bag 7 in fluid communication with the treatment bag 2 via a transfer tubing 8, said filter unit 12 being arranged on said transfer tubing 8.

In this case, during the transfer of the biological fluid from the treatment bag 2 into the transfer bag 7, the particles 5 are retained by the barrier means 6, without reaching the transfer bag 7. Thus, the treated biological fluid collected in the transfer bag 7 is free of particles.

The filter unit 12 comprises a flexible, rigid, or semi-rigid shell within which the porous membrane forming the barrier means 6 is disposed. The filter unit further comprises an inlet orifice 13 and an outlet orifice 14, the filtration medium delimiting an inlet compartment in communication with the inlet orifice 13 and an outlet compartment in communication with the outlet orifice 14.

Advantageously, the filtration medium is held in the filter unit by means of a flexible, sealed frame.

The filtration medium is composed solely of one or more porous membranes.

Advantageously, the filtration medium further comprises one or more layers of a hydrophilic porous material in order to prevent clogging of the porous membrane and/or in order to filter other substances from the biological fluid.

Even more advantageously, the filtration medium is adapted to remove leukocytes from blood or a blood component.

In particular, the filtration medium further comprises, upstream of the porous membrane(s), one or more layers of a hydrophilic porous material each having an average pore size greater than the pore size of the porous membrane.

In particular, the hydrophilic porous material is a spunbonded non-woven fabric or melt-blown fibre.

For example, the filtration medium comprises, upstream of the porous membrane, several layers of non-woven fabric having an average porosity of between 8 and 12 μm.

Such a filter unit is advantageously configured to additionally remove cellular constituents, such as leukocytes, from blood plasma. A particular example of such a filter unit is described in EP 0 953 361 A1.

In the case in which the set of particles 5 binds anti-A and/or anti-B antibodies in the blood plasma, the system comprising such a porous membrane has an application for obtaining a universal leukocyte-depleted plasma for transfusion.

According to a second aspect, a method for treating a biological fluid such as blood or a blood component by selective removal of a target substance using a system according to the first aspect of the invention will now be described. The system further comprises a transfer bag 7 in fluid communication with the treatment bag via a transfer tubing 8.

The method comprises the steps of:
introducing the biological fluid to be treated into the treatment bag 2 containing the set of particles 5 having an affinity for said target substance,
contacting said biological fluid with said set of particles 5 for a time sufficient to bind the target substance to said particles, so as to obtain a mixture of treated biological fluid from which the target substance has been removed and particles bound or not to the target substance,
transferring said mixture from the treatment bag 2 to the transfer bag 7 via the transfer tubing 8, the barrier means 6 of said system preventing the set of particles from passing into the transfer bag, so as to obtain the biological fluid thus treated separated from said set of particles, and
collecting the biological fluid thus treated and separated from the set of particles in the transfer bag.

The biological fluid is in particular blood previously collected from a donor or a blood component derived from blood collected from a donor and separated by, for example, filtration and/or centrifugation from other blood components.

In particular, in the case in which the set of particles has an affinity for anti-A and/or anti-B antibodies, the method is used to obtain a universal particle-free plasma ready for transfusion.

Preliminary example: retention of particles by a porous membrane

In order to test the ability of a porous membrane according to the invention to retain small particles, the following test was conducted.

A filter unit was made, comprising a filtration medium consisting of, from upstream to downstream: a layer of spunbonded polyester non-woven fabric, four layers of melt-blown polypropylene non-woven fabric each having a basis weight of about 40 g/m² and an air permeability of about 110 1/m²/s, a layer of hydrophilically treated polyvinylidene fluoride membrane having a porosity of 0.65 μm, a layer of melt-blown polypropylene non-woven fabric with a basis weight of approximately 40 g/m² and an air permeability of approximately 110 1/m²/, and a layer of spunbonded polyester non-woven fabric. The filtration area is approximately 20 cm².

A bag was filled with a 250 ml suspension of water for injection and ultrafine cellulose particles (5 mg/ml), ellipsoidal in shape with an approximate size of 2.2 μm×8 μm.

The contents of the bag were passed through the filter bag and the number of particles after filtration was determined using a liquid particle counter.

TABLE 1

| | | Average | Average | Average |
|---|---|---|---|---|
| | Volume of | number of | number of | number of |
| | water | particles > | particles > | particles > |
| | analysed | 5 μm | 10 μm | 25 μm |
| Bag | 120 ml | 87.86 per ml | 7.64 per ml | 0.08 per ml |

These results are in accordance with the American Pharmacopeia (volume 6, <788>Particulate matter in injections, USP 42, edition 2019, 6942-6946) and the European Pharmacopeia (10.0, Section<2.9.19>Particulate contamination: Sub-Visible particles, 360-362, 04/2011: 20919), which stipulate that for injectable preparations with a volume of more than 100 ml, the following conditions must be met:

TABLE 2

| Volume of | | Number of | | Number of | |
|---|---|---|---|---|---|
| parenteral | Size of | particles per ml | | particles per ml | |
| preparation | particles | US pharmacopeia | | EP pharmacopeia | |
| >100 ml | ≥10 μm | 25 | per ml | 12 | per ml |
| | ≥25 μm | 3 | per ml | 2 | per ml |

Example: Removal of Anti-B Antibodies from Group O Blood Plasma

Material

750 mg of ultrafine cellulose particles grafted with a group B antigenic oligosaccharide, ellipsoidal in shape and approximately 2.2 μm×8 μm in size were introduced into a PVC bag.

The filter bag used in this example is identical to that described in the preliminary example above.

Plasma Treatment

Approximately 250 ml of group O plasma was obtained by centrifugation of one unit of group O whole blood (approximately 500 ml). After introduction of the plasma into the PVC bag containing the particles, the PVC bag was shaken horizontally for approximately 30 minutes and then the contents were filtered by gravity through the filter bag.

Analyses

Immunoglobulin G (IgG) and immunoglobulin M (IgM) antibody titres were determined in the initial plasma, that is to say before treatment, and in the final plasma, that is to say after filtration through the filter bag.

The determination of the antibody titre was performed by a direct agglutination test for IgM titration and an indirect agglutination test for IgG titration.

Plasma samples (initial and final) were diluted in PBS buffer by a factor of 2 (1/1, 1/2, .. . . , 1/64) and contacted with red blood cells of blood group B. The titre returned was the last dilution in which red blood cell clusters due to the presence of blood antibodies remained visible.

The results are shown below.

TABLE 3

| | Initial antibody | Final antibody | Titration |
|---|---|---|---|
| Titre | titre | titre | gain |
| IgM | 1/128 | 1/2 | 6 |
| IgC | 1/64 | 1/2 | 5 |

Biochemical assays of plasma factors were also carried out. The results are shown in Table 4 below.

TABLE 4

| | Non-treated plasma | Plasma after filtration |
|---|---|---|
| Factor Vc | 58% | 57% |
| Factor VIIIc | 31% | 30% |
| Factor XIc | 85% | 78% |
| Fibrinogen | 2.91 g/l | 2.78 g/l |
| Prothrombin | 79% | 79% |

In this table, the concentrations of coagulation factors factor V, factor VIII, factor IX and prothrombin are expressed as a percentage of a normal human plasma standard. The assays are chronometric assays.

Filtration has no impact on the plasma factors tested: prothrombin, fibrinogen, factor V, factor VIII and factor XI.

1. A system for treating a biological fluid such as blood or a blood component by selective removal of a target substance, comprising a treatment bag provided with at least one inlet orifice and at least one outlet orifice, said treatment bag containing a set of particles having an affinity for the target substance, said set comprising small particles having a size of less than 15 μm, characterised in that the system further comprises a barrier means composed of at least one porous membrane made of a hydrophilic material, the pores of said membrane being calibrated to a size suitable for preventing the passage of said small particles, namely less than 3 μm.

2. The system according to claim 1, wherein the hydrophilic material is selected from naturally hydrophilic materials or plastics-based materials that have been made hydrophilic.

3. The system according to claim 1, wherein the hydrophilic material is selected from polymers and/or copolymers based on polypropylene, polyester, polyamide, high- or low-density polyethylene, polyurethane, polyvinylidene fluoride, or cellulose-based products such as cellulose acetate and its derivatives.

4. The system according to claim 1, further comprises a transfer bag in fluid communication or intended to be put in fluid communication with the treatment bag via a transfer tubing.

5. The system according to claim 1, wherein the barrier means is disposed inside the treatment bag.

6. The system according to claim 5, wherein the barrier means is in the form of a pouch enclosing said set of particles, said pouch being composed of said porous membrane.

7. The system according to claim 6, wherein the pouch is a double-walled pouch, each of the walls being composed of the porous membrane.

8. The system according claim 6, wherein the pouch comprises a first and a second peripheral seal spaced apart from each other.

9. The system according to claim 6, wherein the pouch is sealed.

10. The system according claim 6, wherein the pouch has an opening, said opening being in fluid communication with the inlet orifice of the treatment bag.

11. The system according to claim 5, wherein the barrier means is in the form of a wall composed of said porous membrane, said wall being arranged within the treatment bag so as to delimit an inlet compartment in fluid communication with the inlet orifice of the treatment bag and an outlet compartment in fluid communication with the outlet orifice of the treatment bag, said inlet compartment of the treatment bag enclosing said set of particles.

12. The system according to claim 11, wherein the wall comprising the porous membrane is held in a sealed frame.

13. The system according to claim 1, wherein the barrier means is in the form of a filter unit, said filter unit enclosing a filtration medium composed of at least said porous membrane.

14. The system according to claim 13, further comprising a transfer bag in fluid communication with the treatment bag via a transfer tubing, said filter unit being arranged on the transfer tubing.

15. The system according to claim 13, wherein the filter unit comprises an inlet orifice and an outlet orifice, the filtration medium delimiting an inlet compartment in communication with the inlet orifice and an outlet compartment in communication with the outlet orifice.

16. The system according to claim 13, wherein the filtration medium further comprises one or more layers of a hydrophilic porous material each having an average pore size greater than the average pore size of the porous membrane.

17. The system according to claim 16, wherein the hydrophilic porous material is a spunbonded non-woven fabric or a melt-blown fibre.

18. The system according to claim 13, wherein the filtration medium is able to remove leukocytes from blood or a blood component.

19. A method for treating a biological fluid such as blood or a blood component by selective removal of a target substance using a system according to claim 4, comprising the steps of:
introducing the biological fluid to be treated into the treatment bag containing the set of particles having an affinity for said target substance,
contacting said biological fluid with said set of particles for a time sufficient to bind the target substance to said particles, so as to obtain a mixture of treated biological fluid from which the target substance has been removed and particles bound or not bound to the target substance,
transferring said mixture from the treatment bag to the transfer bag via the transfer tubing, the barrier means of said system preventing said set of particles from passing into the transfer bag, so as to obtain the biological fluid thus treated separated from said set of particles, and
collecting the biological fluid thus treated and separated from the set of particles in the transfer bag.
